(19) United States
(12) Patent Application Publication
(10) Pub. No.: US 2003/0113756 A1
(43) Pub. Date: Jun. 19, 2003

(54) Methods of providing customized gene annotation reports

(76) Inventor: Lawrence Mertz, Gaithersburg, MD (US)

Correspondence Address: MORGAN LEWIS & BOCKIUS LLP, 1111 PENNSYLVANIA AVENUE NW, WASHINGTON, DC, 20004, US

(21) Appl. No.: 10/197,264

(22) Filed: Jul. 18, 2002

Related U.S. Application Data
(60) Provisional application No. 60/305,885, filed on Jul. 18, 2001.

(51) Int. Cl. C12Q 1/68; G06F 17/60; G06F 19/00; G01N 33/48; G01N 33/50

(52) U.S. Cl. 435/6; 702/20; 705/3

(57) ABSTRACT

The invention relates to a method of allowing a customer, such as a physician or biomedical researcher, to quickly access information about a gene or genes, such as the relative expression level of genes in a variety of tissues and biological samples. The method thus allows the customer to obtain valuable information relating the gene to altered physiological states and the study of diseases. The method allows a customer to query a database and/or databases that store such information to produce or receive a customized gene annotation report. The customer does not have to be a subscriber to the database, but can be a one time user of the database.

### CROSS-REFERENCE TO RELATED APPLICATIONS

This application claims priority from U.S. provisional application Serial No. 60/305,885, filed Jul. 18, 2001, the disclosure of which is herein incorporated by reference in its entirety.

### FIELD OF THE INVENTION

The invention relates to business methods that allow a customer, such as a physician or biomedical researcher, to quickly access information about a gene or a set of genes. The methods allow the customer to obtain any relevant functional, structural or genomic information pertaining to one or more genes, such as their relative expression levels in a variety of tissues and biological samples. The methods thus allow the customer to obtain valuable information relating the gene or genes to altered physiological states and the study of diseases. The customer may query a database and/or databases that store such information, and the methods of the invention make available to the customer a customized gene annotation or gene expression report. The customer may also have the ability to select from one or more report content options for the generation of a customized and unique report designed to suit individual needs. The customer may or may not be a regular subscriber to any of the privately owned databases from which the information may be derived.

### BACKGROUND OF THE INVENTION

A wealth of sequence information is now available in sequence databases, both public and private. The advantage of this abundance of data is that better drug treatments will be possible as new drug targets and protein therapeutics are identified and characterized. In addition, small differences in the genetic makeup of individuals, or genotype, result in different physical characteristics, or phenotypes, with the consequence that drugs may help some people but may end up harming others. With knowledge of how different genotypes affect the function of drugs, treatment regimens can potentially be customized based on genetic information associated with a specific patient.

One type of data that is of particular interest is gene expression data. Gene expression reflects how a cell is functioning and how it is responding to its environment. For example, certain genes will be more or less active in a diseased cell than in a healthy cell of the same type. Thus, gene expression data can be used by a physician to aid in the diagnosis and treatment of a disease state. In the area of drug discovery, researchers can develop innovative drugs that prevent or treat the disease by finding compounds that affect these over- or under-expressed genes. Moreover, the time, cost and risk associated with drug discovery and development can be reduced if the expression levels of genes that play roles in disease-associated pathways are known.

Many disease states may be characterized by differences in the expression levels of various genes either through changes in the copy number of the genetic DNA or through changes in levels of transcription (e.g., through control of initiation, provision of RNA precursors, RNA processing, etc.) of particular genes. For example, losses and gains of genetic material play an important role in malignant transformation and progression. Furthermore, changes in the expression (transcription) levels of particular genes (e.g., oncogenes or tumor suppressors), serve as signposts for the presence and progression of various cancers.

Devices and computer systems have been developed for collecting information about gene expression or expressed sequence tag (EST) expression in large numbers of tissue samples. For example, PCT application WO 92/10588, incorporated herein by reference for all purposes, describes techniques for sequencing or sequence checking nucleic acids and other materials. Probes for performing these operations may be formed in arrays according to the methods of, for example, the techniques disclosed in U.S. Pat. Nos. 5,143,854 and 5,571,639, both incorporated herein by reference for all purposes. Further, computer-aided techniques for monitoring gene expression using such arrays of probes have been developed as disclosed in EP Pub. No. 0848067 and PCT publication No. WO 97/10365, the contents of which are herein incorporated by reference.

With DNA microarray technology one can easily collect large amounts of data to indicate which genes or ESTs are regulated upwards or downwards during various disease states, following various pharmacological treatments, or following exposure to a variety of toxicological insults. However, while the quantity of data that one can gather with these techniques is very large, it is often out of context. The relevance of gene expression data is often determined by its relationship to other information within the context of the current analysis. For example, knowing that there is an increased expression of a particular gene during the course of a disease is important information. In addition, there is a need to correlate this data with various types of clinical data, for example, a patient's age, sex, weight, stage of clinical development, stage of disease progression etc. What is needed in the field is a way to correlate the vast amounts of gene and EST expression data that one can obtain using DNA microarrays with the corresponding clinical data from the samples that are tested.

Another downside of the wealth of information now available is that the sheer quantity of data is overwhelming to the individual researcher, who often does not know how to maximize its usefulness. This is complicated by the fact that such information derived from several sources is not assembled in a coordinated fashion. Further, the various sources often provide conflicting information. The wealth of information also presents a challenge to pharmaceutical and biotechnology companies looking for drug targets where such genomics initiatives often present more targets than can be characterized. This in turn often leads to the investigator manually restricting the data set in ways which leave out potentially useful patterns of gene expression.

For instance, current sample-based analysis methods for gene expression data involve manual curation of sample sets. Investigators must begin an analysis with a specific goal (e.g. ‘today I will investigate Alzheimer's disease’) in mind and build their sample sets accordingly. This method biases the resulting analyses towards the initial goal of the investigator and leaves potentially interesting patterns undiscovered and obscured simply because the investigator did not have time to manually exhaust all potential analysis routes through the available data (e.g. discovering a gene regulated in Alzheimer's disease is interesting; finding a gene regulated across all known degenerative neural diseases is potentially far more useful).

### SUMMARY OF THE INVENTION

In light of the current situation, there is a need in the bioinformatics arena to allow users, such as physicians, biomedical researchers, and even laypersons, to access one or more private databases and obtain gene annotation or gene expression reports without having to subscribe to each private database containing such information. The method of the instant invention allows even a one-time customer access to such information. Further, the method of the invention avoids the problems of the prior art by allowing the user to define more general sample relationships in which he or she is interested and automate the creation of all possible valid sample sets defined by these general relationship parameters.

The present invention satisfies the above described needs by providing a means for customers to access systems that correlate normal and diseased tissues or cell lines from humans and experimental animals with critical clinical findings, improving target selection and prioritization. In addition, depending on preference, the customer may extend the systems available to correlate the effects of medication on tissue samples (by comparing non-treated tissues versus treated tissues in a b-tree sorted by tissue and then by medication). In the same fashion, effects due to patient secondary diagnosis, age, race, gender, date of birth, date and/or cause of death and a myriad of lifestyle attributes (such as drug use, smoking, alcohol consumption, exercise habits, diet profile, sleeping habits, etc) and clinical diagnostic data (e.g. cholesterol levels, hematocrits, white blood cell counts, etc.) can be compared and presented within a single report within the framework provided by the present invention.

In addition, the business methods of the present invention utilize a system that has the capability to examine the effects of therapeutic and prophylactic compounds on human and animal tissues or cell lines. One can easily study the mechanism of action of therapeutic compounds and the characteristics of experimental model systems by comparing the gene expression data with known therapeutic and experimental parameters. Similarly, the present invention provides for the customer access to a system that allows one to examine the affects of toxic compounds on tissues and cells in both a pre-clinical and clinical setting.

In one aspect, the invention provides a method of providing one or more gene annotation reports to a customer comprising: (a) receiving at least one gene identifier for a gene from a customer; (b) interrogating one or more databases with the gene identifier; (c) producing a gene annotation report for the gene; and (d) forwarding the gene annotation report to the customer. In some embodiments, the gene annotation report is provided through a gene annotation database, and depending on the query received from the customer, a gene expression database such as a microarray platform. In some embodiments, the gene annotation database uses an algorithm employing a hierarchical method for organizing biological samples for analysis using a b-tree and query grammar to manage and explore gene expression and related data as disclosed in application Serial No. 60/331,182, filed Nov. 9, 2001, No. 60/388,745, filed Jun. 17, 2002, and No. 60/390,608, filed Jun. 21, 2002, which are herein incorporated by reference in their entireties.

### BRIEF DESCRIPTION OF THE DRAWINGS

FIG. 1 is a flow chart that provides an overview of the method of preparing and providing a gene annotation report request by the customer. It lists the types of input information as well as the process that may be used to query gene annotation databases to generate the report.

FIG. 2 shows one example of a gene annotation report which includes at least one or more various annotation categories. It includes, but is not limited to, DNA sequence information and genomic mapping, gene expression information in normal and diseased tissues as well as demographic detail, metabolic pathway information, proteomic information, splice variant and SNP information. Information regarding commercially available clones and patents that have been filed and issued concerning query nucleic acid and protein sequences is also included.

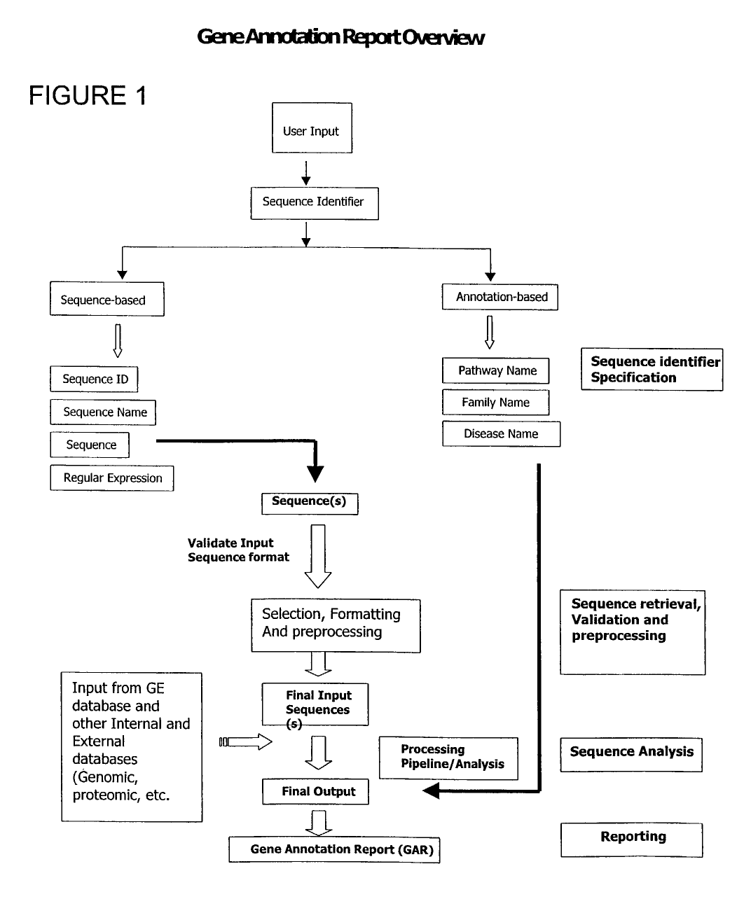

### DETAILED DESCRIPTION OF THE INVENTION

Definitions

Informatics is the study and application of computer and statistical techniques to the management of information. Bioinformatics is the use of these techniques for the management of biological information and includes the development of methods to search databases quickly, to analyze nucleic acid and/or protein sequence information, to compile and analyze gene and protein expression data, and to correlate different pieces of data.

Annotation is the process of attaching comments to data labels and making connections to related data. The comments may include any and all information that can be known about a gene or genes. Sequence information may include the library in which a given sequence was found or descriptive information about related cDNA(s) associated with the sequence. Expression information may include tissues in which the gene is normally expressed, disease states associated with up- or down-regulation of the gene, gene expression levels at various stages of a disease process, or expression levels during various developmental stages. Additional genomic information may describe biological function, biological pathways in which the gene is involved, single nucleotide polymorphisms, splice variants, etc.

Gene expression is the process by which genes are converted into the structures present and operating in the cell. Expressed genes include those that are transcribed into mRNA and then translated into protein and those that are transcribed into RNA but not translated into protein, e.g., transfer RNA and ribosomal RNA.

Electronic Northern, or eNorthern™, refers to a report concerning the use or mining of a sequence or gene expression databases to identify the relative levels of messenger RNA expressed in different cells or tissues. One can use the information to find genes that are expressed only in specific tissues or during specific stages of cell differentiation and development. Electronic Northerns may also identify differentially expressed genes associated with altered physiological conditions, such as disease states.

Sequence alignment is part of the process of comparing sequences for similarity, and may include the introduction of phase shifts or gaps into the query sequence or the sequences contained in the databases being searched in order to maximize the similarity between the sequences. Global alignment is the alignment of two sequences over their entire length whereas local alignment is the alignment of a portion of two sequences.

Homology refers to the evolutionary relatedness of sequences.

A disease state or altered physiological state refers to any abnormal biological state of a cell. A disease state may be the consequence of infection by a pathogen, such as a virus, bacteria, or fungus. It may also result from the effects of an agent such as a toxin or carcinogen. An altered physiological state may result from brief or prolonged exposure to a toxic substance, extreme environmental conditions, or possibly from the administration of pharmaceuticals. In addition, genetic disorders, wherein one or more copies of a gene are altered or disrupted, may also lead to an altered physiological state or disease state, and include, among others, sickle cell anemia, thalassemia, and Tay-Sachs disease.

A biological pathway is a collection of cellular constituents related in that each cellular constituent of the collection is influenced according to some biological mechanism. The cellular constituents making up a particular pathway can be drawn from any aspect of the biological state of a cell. Biological pathways include well-known biochemical pathways, for example, pathways for protein and nucleic acid synthesis. Nutrient metabolism is also a well-known biological pathway. Others include cell surface and intracellular signaling cascades, transcriptional activation mechanisms, secretory mechanisms, changes in cell membrane potential, differentiative and other similar cell response control pathways.

A gene annotation database is a database through which information from multiple databases, public or private, may be accessed, assembled, and processed.

Methods of the Invention

The invention relates to a method of providing a gene annotation and/or gene expression report to a customer. In some embodiments of the invention, the customer submits a gene identifier to the gene annotation database provider and requests a gene annotation and/or gene expression report. A gene identifier is any relevant query information, including but not limited to nucleotide sequences, amino acid sequences, sequence database identifiers, for example, but not limited to GenBank or Unigene identifiers, gene names or symbols and/or protein names or symbols. A gene annotation report is a report containing structural and functional genomic and/or proteomic information with respect to the gene identifier and relevant links to reagents and/or public information relating to the gene identifier. A gene expression report contains functional genomic and/or proteomic information with respect to the gene identifier.

The gene annotation database allows access to one or more databases, public or private. These databases are then interrogated using the gene identifier(s), a customized gene annotation report is produced, which is then forwarded to the customer. The customer may or may not be a full time subscriber to any of the databases described. Thus, gene annotation reports are made available to many different customers, such as physicians, biomedical researchers, or even laypersons, who do not have the resources or need to subscribe to the various private biological databases.

Customers may also request gene expression data relating to the expression of one or more genes in one or more tissues, in normal or disease states, using the database and methods of the invention. Using the services of the invention, a customer may correlate the expression of sample gene sequences or ESTs to particular tissue types. Various tissue types may correspond to different diseases, states of disease progression, organs, species, etc. A customer may also obtain comparative data sets in order to analyze the affects of toxic compounds on tissues and cells in both a pre-clinical and clinical setting, or to monitor the progression of different diseases based on a patient's gene expression data. Other applications include development of pharmaceuticals, cosmetics, food additives, pesticides, herbicides and other biological-acting materials based on the genomics information supplied.

Annotation and Expression Reports

The gene annotation and/or expression report may contain various types of information known about the gene or genes depending on the report content options specified by the customer. For example, the report may contain information regarding the identity of cells or tissues in which the gene(s) are expressed along with the relative level of expression. The report may also contain information concerning the disease state of the cell or tissue in which the gene was expressed and/or physiological characteristics of the cell or tissue. Information concerning the patient from whom the cell or tissue was derived may be included, such as clinical, ethnic, race, age, gender and other relevant demographic or personal data (including, for instance, secondary diagnoses, family history and lifestyle attributes, such as drug use, smoking, alcohol consumption, exercise habits, diet profile, sleeping habits, etc.). Pertinent clinical information may include diagnostic data, e.g. cholesterol levels, hematocrits, ankle brachial index, abdominal aortic aneurysm, carotid ultrasound scan, thyroid ultrasound scan, osteoporosis screening, body composition, blood and pulse pressure, oxygen saturation, hearing screening, vision screening, urine analysis, blood studies (PSA, blood count, white blood cell count, chemistry panel, lipid panel, triglycerides and risk ratio, thyroid blood test, C-reactive protein, fibrogen, homocysteine, CEA, CA-125, hormones, CT scans, etc).

The gene annotation report may also contain genomic and/or proteomic information, such as gene expression, single nucleotide polymorphisms (SNP), splice variants, the locations of introns and exons, functional domains and/or biological pathways in which the gene is involved. Related gene homologues and orthologues in other eukaryotic and prokaryotic organisms may be identified. In addition, other related information can be provided such as the identities of homologous and related gene family members with similar gene expression metrics, chromosomal and genomic DNA mapping information, EST mapping and clustering information. The report may also include a listing of biological relationships pertaining to the particular query sequence, such as the identities of proteins and peptides that have a receptor-coreceptor relationship with a query protein or a protein encoded by a query gene, or known antibodies or antibody fragments that are known or would be predicted to bind to regions of the query protein. To this end, WO 00/15847 discloses methods of applying inference rules to infer missing information and define biological relationships in a method of genomic information analysis, and is herein incorporated by reference in its entirety.

The report may also relay information pertaining to families or subsets of genes related to a query gene identifier, such as those selected from the group consisting of families or subsets of genes involved in one or more biological or signal transduction pathways, genes encoding homologous proteins, genes encoding proteins that share conserved motifs, genes that encode the top pharmaceutical drug targets and genes involved in a specified disease. Some examples of genes encoding proteins that share conserved motifs include, but are not limited to the group consisting of genes encoding G-protein coupled receptors, kinases, antibodies and DNA binding proteins.

Background information on the biological function of the query DNA sequence may be included in the report along with information on whether a clone, cell line, transgenic animal, or other reagent containing the customer query sequence can be purchased from a biorepository or other supplier. Subscriptions may also be made available for individuals or companies offering gene related products who would like to advertise or ensure that users of the databases of the invention are made aware of the subscriber's product or products when they have a useful relation to the query. Further useful information may also be included depending on the preferences set by the user, including treatment information for specific diseases, including the identity and potentially the affinity of different pharmaceuticals and compounds, the locations of doctors who specialize in treating the indicated disease, relevant clinical trials, what tissues are affected or perhaps side effects relating to specific pharmaceuticals, and the like.

Query Methods

The embodiments of the present invention allow a customer, such as a physician, researcher, or layperson to query private and public databases to assemble gene annotation and/or expression reports. Both the request and the delivery of a customized gene annotation and/or expression report to the customer may be done electronically, such as over the internet or via e-mail, a modem-to-modem download, or by use of a computer-readable storage medium or by facsimile, mail, or any other means. Customers may also purchase limited databases for personal use, i.e., from a local CDROM drive.

In exchange for the provided gene annotation report, the customer may be automatically invoiced for payment. Such payment can be provided through an on-line process such as credit card payment or the like. It is also possible that some customers who request gene annotation or expression reports on a regular basis, or more frequently than a “single-use” customer could have an account set up with a customer ID number and/or password which would automatically generate the appropriate invoice and automatic payment procedure.

In one embodiment, a customer may submit an alert request, so that if new information becomes available pertaining to a sequence or disease of interest, a notification or alternatively the information itself is forwarded to the customer. Automatic notification is a feature provided in many databases and is usually based on a query which is re-run periodically at the database. If any new data becomes available, the user of the database is notified.

The gene identifier submitted by the customer may for example be a nucleotide sequence, an amino acid sequence, a sequence database identifier, a gene name and/or symbol and/or a protein name and/or symbol. When seeking to obtain information relating to the identities of known genes or gene sets, gene query information may be submitted such as the name of a disease state, or a tissue or organ type. If a sequence database identifier is used, such an identifier may be a GenBank accession number or an Affymetrix™ fragment identifier. It is also be possible for a customer to submit a reference citation referring to one or more genes and request information pertaining to the gene discussed in the reference.

The customer may also submit more than one gene identifier to the database at the same time. Such examples of multiple gene submissions include, but are not limited to, genes whose protein products constitute a known or proprietary biological or signal transduction pathway, a family of genes that contain a common domain or motif feature such as G-protein coupled receptors, protein kinases, antibodies or DNA binding proteins, genes encoding protein homologues, genes associated with a specific disease and genes whose protein products are targets for the top 100 pharmaceuticals in the marketplace, for instance. Any gene compilation known in the art may be submitted as a gene identifier in the methods of the invention.

The step of interrogating a gene annotation database with the gene identifier or gene query may consist of comparing the gene identifier or gene query to information in the database. If the gene identifier is an accession number, the comparison step may comprise locating the accession number in the gene annotation database. If the gene identifier is a sequence, the comparison may comprise the step of comparing the sequence to sequences in the gene annotation database. Such comparisons may be done through the use of sequence alignment algorithms or homology searches, and may be performed singularly or repetitively at various levels of stringency, for instance until a match is found.

One embodiment of the invention utilizes a set of algorithms and database-related scripts and commands that are executed to generate such reports. A suitable algorithm that may be used to provide the reports of the present invention is disclosed in copending application Serial Nos. 60/331,182, 60/388,745 and 60/390,608, which are herein incorporated by reference. Exploring gene expression data involves mechanisms for integrating gene expression data across multiple platforms and with detailed sample and gene annotations. The algorithm disclosed in application Serial Nos. 60/331,182, 60/388,745 and 60/390,608 uses a hierarchical method for organizing biological samples for analysis using a b-tree and a query grammar to manage and explore gene expression and related data. In this way, samples are associated with attributes that describe properties useful for gene expression analysis, for example, sample structural and morphological characteristics (e.g., organ site, diagnosis, disease, stage of disease, etc.) and donor data (e.g., demographic and clinical record for human donors, or strain, genetic modification, and treatment information for animal donors).

For instance, application Serial Nos. 60/331,182, 60/388,745 and 60/390,608 disclose a method for analyzing gene expression data, the method comprising: (a) organizing the data into a b-tree comprising a plurality of levels, each level comprising a plurality of leaf nodes; (b) defining a plurality of attributes for filtering the data at each level of the b-tree; (c) distributing the data among the plurality of leaf nodes according to the plurality of attributes; (d) grouping the leaf nodes according to their corresponding attributes; (e) defining a control sample set and an experimental sample set; (f) performing a t-test comparing the experimental sample set with the control sample set; and (g) generating a table of t-test results. The plurality of attributes may comprise structural and morphological characteristics of gene expression data, for instance, organ site, diagnosis, disease, stage of disease, demographic and donor data. Such donor data may be from either a human donor and include data such as height, weight, race, date of birth, cause of death, age at death, secondary medical conditions, exercise habits, diet profile, sleeping habits, smoking habits, alcohol habits, drug habits, etc., or may be from an animal donor and include strain, genetic modification and treatment information, for instance. The present invention serves as a bridge between such an algorithm and medical or other types of consumers, organizing and presenting the results in customized reports according to user preference.

Other algorithms are known in the art and may be used to provide various aspects of the customer-oriented reports of the invention. For instance, algorithms exist for predicting coding regions in eukaryotic genomes, such as the gene prediction programs GRAIL and GRAIL II, Uberbacher et al., Proc. Natl. Acad. Sci. USA 88(24):11261-5 (1991); Xu et al., Genet. Eng. 16:241-53 (1994); Uberbacher et al., Methods Enzymol. 266:259-81 (1996); GENEFINDER, Solovyev et al., Nucl. Acids. Res. 22:5156-63 (1994); Solovyev et al., Ismb 5:294-302 (1997); and GENSCAN, Burge et al., J. Mol. Biol. 268:78-94 (1997), and DICTION (see U.S. Patent Application 2002/0048763), which are all incorporated by reference in their entireties. Any other suitable algorithm existing in the art or to be designed in the future may be used to identify and define relationships among bits of genomics information existing in the one or more databases utilized to generate the reports of the present invention.

In addition, the algorithms employed by the programs blastp, blastn, blastx, tblastn and tblastx may be used (Karlin, et al., Proc. Natl. Acad. Sci. USA 87: 2264-2268 (1990) and Altschul, S. F. J. Mol. Evol. 36: 290-300(1993), fully incorporated by reference), which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. (Nature Genetics 6: 119-129 (1994)) which is fully incorporated by reference.

It is envisioned that direct sequence hits of the query sequence can be identified that are homologous to DNA probe sequences or similar elements arrayed on a chip or similar gene expression analysis platform. In addition, indirect sequence hits can also be identified that pertain to those that bear no direct homology to the query sequence, but have homology to sequences that map to 5′ and 3′ regions of the query sequence through contig assembly of related DNA sequences. It is also possible that other biological databases such as proteomic databases could be queried and a report could be generated, based on the reading frame is provided for a particular DNA coding sequence, or by analyzing the sequence in all three frames.

An aspect of some embodiments of the invention is that the user may specify that inferences are based on a meaning of terms (semantics), rather than on an exact word match. For example, semantics could be employed such that the terms IL-2 and interleukin 2 would be interpreted as the same concept. Alternatively or additionally, the broadening of a term may be applied at a query construction stage (‘query expansion’). The broadening for a particular database may be limited to exclude terms which are known not to be in use in the database and/or exclude terms which occur far too frequently to be meaningful. In some embodiments, semantic mapping may be used as described in WO 00/15847, which is herein incorporated by reference.

When semantic mapping is applied during query formulation, it may be used to define a larger set of related key words, i.e., to broaden a term, like heart tissue to include myocardium, papillary muscle, etc. Alternatively or additionally, semantic mapping may be used when broadening a query, for example by suggesting higher levels of abstractions. For example, “intestinal muscle” may be broadened to “smooth muscle.” Alternatively or additionally, the semantic mapping may be applied when adapting the query to a particular database. Alternatively or additionally, the semantic mapping is applied while parsing the results of a query, and may be used to suggest further query terms to the customer. Semantic mapping may be performed by an inference engine as disclosed in WO 00/15847, or alternatively, semantic mapping may be performed using a comprehensive database of biomedical terms, for example, initially populated with content from the Unified Medical Language System (UMLS) knowledge base, available from the National Library of Medicine.

One embodiment of the invention comprises computer database-derived methods for compiling gene expression information in the form of intensity values from nucleic acid array chip or other quantitative or semi-quantitative gene expression analysis methods, such as Q-RT-PCR. This information may then be queried to determine gene expression levels in various biological samples for the purpose of comparing relative gene expression information between various biological samples such as human tissues and cell lines. The report would reveal the expression ranges of genes in a set of biological samples from a population of individuals. Such a report is termed an “Electronic Northern” or an “E-Northern,” and may be comprised of tissue and cell expression information over a variety of samples. It may also contain specific expression information in various disease and altered biological states where relevant, e.g., where differential gene regulation is observed with statistical significance. Such a report would educate the customer as to the scope and variety of diseases the regulation of a certain gene is observed. The method can therefore be used to determine the expression ranges of a gene or genes with respect to an altered biological state versus that of a normal state.

In addition, the method allows the customer, such as a biomedical researcher, to determine the various disease areas where a gene shows differential regulation once the gene has been identified as a marker or potential therapeutic drug target in another related or different disease state. For example, if a researcher has determined that a certain gene is regulated in breast cancer, a query of the expression database can be performed to determine what diseases associated with other tissues demonstrate the same gene regulation. This could significantly impact the discovery of genes that regulate disease processes such as cancers, degenerative diseases, and auto-immune diseases. Moreover, such information would augment the search for genes that can be recruited as markers for disease as well as to search for genes that are drug targets to treat disorders and diseases. Such methods may also be used in the drug or agent screening assays.

Databases

The present invention includes the generation of gene annotation and/or expression reports derived from public and private relational databases such as those containing sequence information and/or gene expression information in various cell or tissue samples. The databases used to generate the gene annotation and/or expression reports may also contain information associated with a given sequence or tissue sample. This may include descriptive information about the gene associated with the sequence information, descriptive information concerning the clinical status of the tissue sample or that of the patient from which the sample was derived. The gene annotation database is thus designed to include and allow access to different informational databases, for instance a sequence database and a gene expression database, and to provide means for analyzing such data so that it may be communicated to the customer in a meaningful format. Methods for the configuration and construction of such databases are widely available, for instance, see U.S. Pat. No. 5,953,727, which is herein incorporated by reference in its entirety.

The databases of the invention may be linked to an outside or external database. In one embodiment, the external database is GenBank and/or the associated databases maintained by the National Center for Biotechnology Information (NCBI—see, http://www.ncbi.nlm.nih.gov). Such databases include UniGene, GeneMap, EST, STS, and SNP Database(s), Online Mendelian Inheritance in Man Database (OMIM™), Diseases and Mutations, and Blast Engine(s), to name a few. Other databases may also be accessed, including databases of the National Library of Medicine (NLM), the Federal Drug Administration (FDA), the National Institutes of Health (NIH), among others. In accordance with an embodiment of the present invention, gene expression data may be generated directly by the supplier of the database of the invention or a collaborator thereof, using the Affymetrix GeneChip® platform, marketed by Affymetrix Corporation of Santa Clara, Calif., and may be represented in the Genetic Analysis Technology Consortium (“GATC”) relational format.

Any appropriate computer platform may be used to perform the necessary comparisons between the gene identifier, sequence information, gene expression information and any other information in the database to generate the gene annotation report. For example, a large number of computer workstations are available from a variety of manufacturers, such has those available from Silicon Graphics. Client/server environments, database servers and networks are also widely available and appropriate platforms for the databases of the invention.

The gene annotation reports of the invention may use the databases to produce, among other things, electronic Northerns that allow the user to determine the cell type or tissue in which a given gene or genes are expressed. As discussed, ENortherns also allow determination of the abundance or expression level of a given gene or genes in a particular tissue or cell.

Use of Gene Annotation and Expression Reports by Physicians

In the clinical setting, a physician must select the most effective and safe drug for a patient, among several drugs. The efficacy and toxicity of a drug to an individual may vary due to a number of factors, such as genetic variation. Thus, a gene annotation and/or expression report may aid the physician in not only selecting the proper therapy for a patient, but also for purposes such as genetic counseling and for prenatal screening and other types of genetic screening, i.e. for inherited diseases or diseases having genetic risk factors, including Alzheimer's disease, Huntington's disease, Parkinson's disease, cancer, arthritis and other autoimmune disorders, etc. Depending on the extent of the information submitted with the query, reports can be generated that provide risks for certain diseases based on the EST, protein, gene or gene sequences provided, cross-referencing demographics, age, gender and secondary medical diagnoses.

In another example, before a physician prescribes and administers a pharmaceutical drug, he/she may want to determine if the patient can be included in a particular racial, ethnic or human population subclass that may possess a high chance of a genomic alteration. Genomic alterations include SNP's, mRNA splice variants, or other alterations for a particular genomic locus that encodes the drug target. The physician could access a gene annotation report to determine if genomic variability is included in that region. Identification of such an alteration associated with the patient profile may help determine the appropriate pharmaceutical regimen.

Another example includes using a combination of gene expression testing and gene annotation reports. After a physician conducts gene expression testing by determining the expression levels of key genes in a given biological sample, he/she may consult gene annotation reports for these genes to determine if there is other information concerning expression of the same genes in disease or altered physiological states. This may assist the physician in determining diagnosis for the disease and achieving proper clinical protocols.

A physician may also want to obtain a gene expression report providing a listing of genes or ESTs showing elevated or reduced expression in particular tissues or disease states, for instance to assist in diagnosis and/or treatment of a particular disease. In such cases, the physician might begin with a query for a sample of genes relating to a particular tissue or disease type, potentially cross-referencing the output results according to other patient characteristics, such as demographics, secondary diseases, age, race, gender, ethnicity, life style attributes, medications, etc.

Use of Gene Annotation and Expression Reports by Laypersons

The general population may also request or use gene annotation report information. Laypersons may wish to conduct their own biomedical research via the internet. For example, if the particular protein target of a drug or therapeutic protein (recombinant or monoclonal antibody) is known, the patient could do more background research on that target to determine if he/she may potentially have a toxic response to a particular drug or pharmaceutical regimen due to the ethnic, racial or population category that he/she may be a member of.

Other non-medical businesses may also make use of the databases of the invention. For instance, by submitting one or more gene sequences or ESTs of a particular client, businesses involved in researching ethnic backgrounds could compare a client's profile to different ethnic population samples in order to trace a client's heritage. This embodiment might be particularly useful for businesses that research family heritages, for example, for adopted clients, or for clients that have little surviving family and who want to know more about their own ethnic background.

The following examples are provided to describe and illustrate the present invention. As such, they should not be construed to limit the scope of the invention. Those in the art will well appreciate that many other embodiments also fall within the scope of the invention, as it is described hereinabove and in the claims.

### EXAMPLES

### Example 1

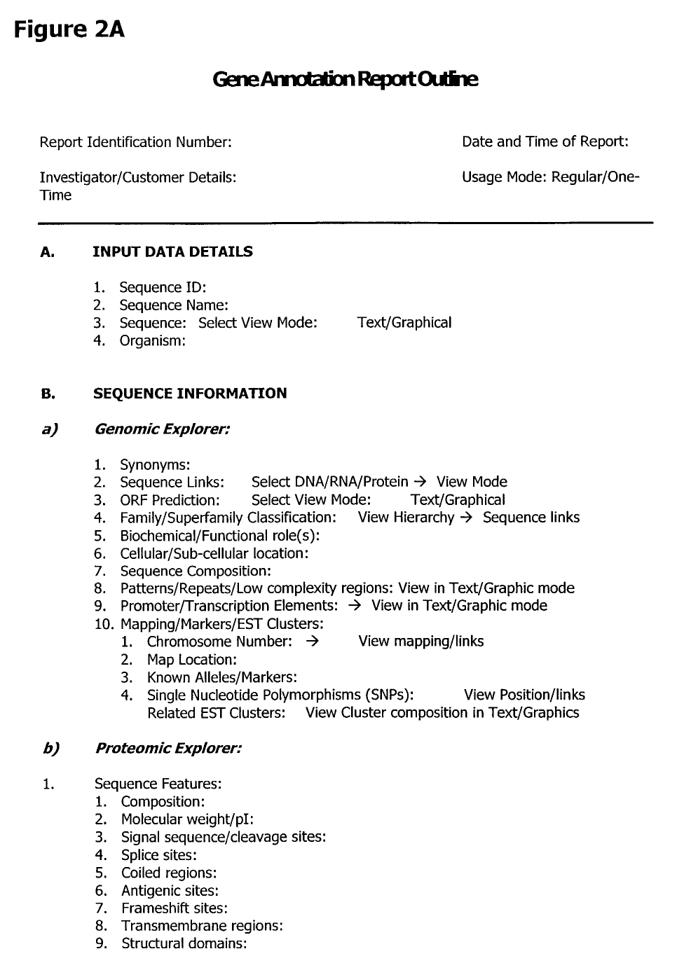

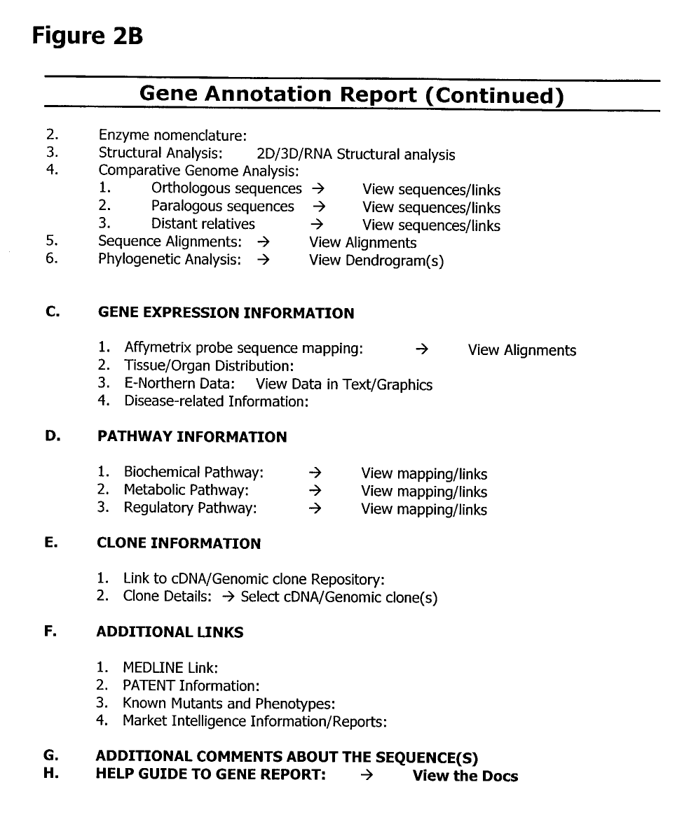

FIG. 2 provides a blank sample Gene Annotation Report showing some of the various categories that a user might include in the report parameters. For instance, depending on the input data, a user may obtain sequence information including synonyms, sequence links, classification, biochemical and/or functional roles, cellular or subcellular location of the expressed protein, sequence composition and regions of interest such as patterns, repeats, low complexity regions, the position and identity of promoter and/or other transcription elements, mapping information including map location, chromosome number, known alleles and/or markers, SNPs and related EST clusters. Proteomics information may also be requested, including composition, molecular weight, the presence and position of signal sequences and other cleavage sites, splice sites, functional and structural domains such as coiled regions and transmembrane domains, antigenic sites and corresponding known antibodies, frameshift sites, enzyme nomenclature, orthologues and paralogues, sequence alignments and phylogenetic analyses. Structural analyses may be included in the form of 2D or 3D structures.

In addition to information relating to the query gene and/or protein sequence, gene expression information may be included, such as tissue/organ distribution in normal and diseased tissues, e-Northern data, and microarray probe sequence mapping, for instance using Affymetrix probe alignments. Biochemical pathway information may also be requested by the customer, as well as information pertaining to available clones, cell lines, transgenic animals, or any other source of the query sequence. Additional links may be provided for the customers convenience, for instance to medline articles, market reports, or to published patents and patent applications, particularly in embodiments where reports are supplied in electronic format. Information pertaining to known mutants and the phenotypes thereof may also be provided.

As can be seen by the exemplary report depicted in FIG. 2, the expression behavior of a gene can be studied over one or many different human disease morphologies and categories to catalog and gauge it's expression with respect to broader human systems biology. For example, it can be more easily determined if a gene or set of genes share similar or divergent expression metrics in multiple related human disease states, such as in cancer morphologies or in inflammatory, or degenerative diseases (for example). Additional information related to gene expression with respect to other clinical parameters such as patient age, race and medication profile (for example) can be combined with such disease profile information to provide a better understand the (combination of) human variables that influence expression of one or more genes.

### Example 2

Tables 1-9 provide examples of gene sequence alignment and expression data that can be included in a Gene Annotation (described in Example 1) and/or Gene Expression report. The data in Tables 1-9 are applicable to a commonly known human extracellular matrix metalloproteinase known as MMP-7. Table 1 includes sequence alignment information for MMP-7 with respect to microarray (Affymetrix GeneChip®) probe sequences from which the gene expression data in Tables 2-9 were generated. Such sequence alignment information could be included in Section B of the Gene Annotation Report Outline shown in FIG. 2. The gene expression data were derived from Gene Logic's BioExpress® Database using data mining algorithms as disclosed in copending application Serial Nos. 60/331,182, 60/388,745 and 60/390,608, herein incorporated by reference, that compile differential gene expression data across a large expanse of clinically-annotated human tissues. Although the gene expression data shown here in Tables 2-9 were obtained from Gene Logic's microarray platform, any other databases and sources of gene expression information could be used to generate the reports of the invention, as described herein and broadly depicted in the report of FIG. 2.

Table 2 provides MMP-7 gene expression data from a panel of normal human tissues. These data could be included in Section C of the Gene Annotation Report Outline (FIG. 2) referred to as an E-Northern. The E-Northern data show MMP-7 to have high expression levels in gall bladder relative to other tissues. Lower, but detectable, expression levels reside in pancreas, prostate, breast and endometrium for example. However, expression appears to be largely absent in GI tract tissues (colon, duodenum, small intestine and rectum), heart (atria and ventricles) and brain regions (cortex of frontal lobe, cortex of temporal lobe, hippocampus). These expression data in normal tissues provide the researcher an overall tissue expression fingerprint across an atlas of human tissues which can help identify target tissues where the query gene is expressed as well as provide a guide to the range of tissues that may be impacted should the protein product of the MMP-7 gene be a drug target. This information can also be used to direct the researcher to tissue models and cell models to study MMP-7 gene expression. For example, a breast or colon tissue-derived cell line would be better than human neuron cell culture.

The data in Tables 3-9 provide the researcher with a relatively comprehensive status of gene expression data for MMP-7 with respect to disease and other relevant human clinical parameters. The differential gene expression data with respect to disease morphology in Table 3 clearly shows significant regulation in several tissue neoplasms and cancers in breast, lung, colon, liver, kidney and myometrium for example. In most cases, it appears that MMP-7 gene expression is significantly upregulated, except for in all breast cancers and in liver cancer. Dramatic upregulation (over 100 fold in many cases) can be observed in ovarian cancer morphologies which is consistent with Tanimoto, H et. al. (The matrix metalloproteinase pump-1 (MMP-7, matrilysin): A candidate marker/target for ovarian cancer detection and treatment; Tumor Biol. March-April 1999; 20(2): 88-98). In aggregate, these disease expression data indicate that MMP-7 may be a candidate marker gene for several tissue cancers and itself, may constitute a drug target since it's upregulation is coincident with cancer morphology.

Table 4 data indicate significant differences in MMP-7 gene expression regulation between stages of several cancers that were identified in Table 3. These data may be used to determine if MMP-7 can be a biomarker to identify and categorize stages of cancer progression. Data in Tables 5-8, provide more information concerning MMP-7 expression in morphologically normal tissues as a function of patient secondary disease, medication status, age and race, respectively. For example, it appears that several different types of patient medication significantly down-regulate MMP-7 expression in the kidney. This information may be pertinent to determining the effect of multiple medications on gene expression, especially if MMP-7 itself is a drug target for a particular medication/therapeutic and patients are taking the other indicated medications listed in Table 6 for kidney.

In this example for MMP-7, no significant gene expression gender differences where found in normal tissues as indicated in Table 9.

TABLE 1
Sequence Identifier Alignment Information

| | | | | | | | | | GB | GB | |
|---|---|---|---|---|---|---|---|---|---|---|---|
| | Hit | | GB Acc. | Match | | | Match | Hit | Match | Match | GB Hit |
| Chip Type | Type | Hit Name | # | % | Score | P-Value | Length | Length | % | Length | Length |
| HG_U95A | Direct | 668_s_at | NM_002423 | 100 | 2401 | 2.30E-103 | 483 | 482 | | | 1127 |

TABLE 2
E-Northern ™ Table for 668_s_at: Gene Expression in Normal Tissues.

| | Global | | | | | | | |
|---|---|---|---|---|---|---|---|---|
| | Present | | | | | Lower | | Upper |
| Fragment | Freq. | Tissue | Morphology | Present | Absent | 25% | Median | 75% |
| 668_s_at | 0.4645 | | | | | | | |
| | | Adipose | Normal Tissue | 7 of 29 | 22 of 29 | 1.48 | 9.12 | 26.95 |
| | | Adrenal Gland | Normal Tissue | 0 of 10 | 10 of 10 | −7.47 | 1.29 | 15.69 |
| | | Breast | Normal Tissue | 68 of 73 | 5 of 73 | 140.20 | 323.60 | 455.36 |
| | | Cervix | Normal Tissue | 77 of 99 | 22 of 99 | 31.18 | 62.40 | 155.10 |
| | | Colon | Normal Tissue | 8 of 140 | 132 of 140 | −5.61 | −0.73 | 6.82 |
| | | Cortex Frontal | Normal Tissue | 0 of 6 | 6 of 6 | −3.77 | 2.33 | 6.60 |
| | | Lobe | | | | | | |
| | | Cortex Temporal | Normal Tissue | 0 of 3 | 3 of 3 | −7.19 | −5.03 | −3.30 |
| | | Lobe | | | | | | |
| | | Duodenum | Normal Tissue | 1 of 33 | 32 of 33 | −7.62 | −0.37 | 4.84 |
| | | Endometrium | Normal Tissue | 26 of 27 | 1 of 27 | 132.50 | 293.25 | 606.63 |
| | | Esophagus | Normal Tissue | 14 of 25 | 11 of 25 | 4.83 | 12.14 | 62.11 |
| | | Gall Bladder | Normal Tissue | 6 of 6 | 0 of 6 | 1529.28 | 2014.44 | 3556.21 |
| | | Hippocampus | Normal Tissue | 0 of 5 | 5 of 5 | −7.04 | −4.23 | 0.90 |
| | | Kidney | Normal Tissue | 65 of 69 | 4 of 69 | 89.03 | 208.95 | 482.87 |
| | | Larynx | Normal Tissue | 3 of 5 | 2 of 5 | 9.81 | 33.06 | 58.72 |
| | | Left Atrium | Normal Tissue | 2 of 128 | 126 of 128 | −3.35 | 2.19 | 8.63 |
| | | Liver | Normal Tissue | 3 of 17 | 14 of 17 | 0.30 | 7.47 | 20.32 |
| | | Lung | Normal Tissue | 62 of 79 | 17 of 79 | 25.81 | 48.41 | 81.76 |
| | | Lymph Node | Normal Tissue | 5 of 11 | 6 of 11 | 6.22 | 16.98 | 302.31 |
| | | Muscles | Normal Tissue | 1 of 42 | 41 of 42 | −10.32 | −2.21 | 4.40 |
| | | Myometrium | Normal Tissue | 98 of 114 | 16 of 114 | 48.26 | 115.05 | 229.48 |
| | | Omentum | Normal Tissue | 6 of 14 | 8 of 14 | 3.53 | 10.58 | 43.41 |
| | | Ovary | Normal Tissue | 9 of 67 | 58 of 67 | −7.70 | −1.22 | 9.11 |
| | | Pancreas | Normal Tissue | 19 of 19 | 0 of 19 | 119.30 | 283.54 | 702.12 |
| | | Prostate | Normal Tissue | 28 of 29 | 1 of 29 | 98.35 | 391.71 | 806.84 |
| | | Rectum | Normal Tissue | 2 of 46 | 44 of 46 | −7.38 | −2.33 | 2.51 |
| | | Right Atrium | Normal Tissue | 4 of 141 | 137 of 141 | −2.52 | 3.00 | 8.38 |
| | | Right Ventricle | Normal Tissue | 0 of 29 | 29 of 29 | −0.28 | 3.66 | 8.48 |
| | | Skin | Normal Tissue | 37 of 53 | 16 of 53 | 18.27 | 44.22 | 112.13 |
| | | Small Intestine | Normal Tissue | 3 of 60 | 57 of 60 | −2.58 | 2.06 | 6.69 |
| | | Soft Tissues | Normal Tissue | 4 of 7 | 3 of 7 | −0.86 | 18.53 | 51.32 |
| | | Spleen | Normal Tissue | 2 of 30 | 28 of 30 | −6.05 | −3.63 | 6.66 |
| | | Stomach | Normal Tissue | 17 of 42 | 25 of 42 | −1.29 | 11.43 | 36.20 |
| | | Thymus | Normal Tissue | 37 of 74 | 37 of 74 | 10.55 | 22.26 | 33.84 |
| | | Thyroid Gland | Normal Tissue | 0 of 18 | 18 of 18 | −5.99 | 1.42 | 5.01 |
| | | Uterus | Normal Tissue | 42 of 46 | 4 of 46 | 55.6 | 177.01 | 356.32 |
| | | WBC | Normal Tissue | 0 of 44 | 44 of 44 | −8.88 | −4.5 | −0.35 |

TABLE 3
Differential Gene Expression with Respect to Disease/Morphology.

| | | | | C¹ | | | | | | | | Fold | |
|---|---|---|---|---|---|---|---|---|---|---|---|---|---|
| Affy ID | Tissue | Disease | Morphology | Mean | C SD | C # | CPC % | E²Mean | E SD | E # | EPC % | Change | T-Stat |
| 668_s_at | BREAST, | MALIGNANT NEOPLASM OF FEMALE | INFILTRATING LOBULAR | 257.60 | 240.85 | 74 | 91.89 | 141.07 | 300.25 | 24 | 66.67 | −1.83 | −1.73 |
| | NOS | BREAST, NOS | CARCINOMA | | | | | | | | | | |
| 668_s_at | BREAST, | MALIGNANT NEOPLASM OF FEMALE | INTRADUCTAL CARCINOMA, NOS | 257.60 | 240.85 | 74 | 91.89 | 76.17 | 72.54 | 7 | 71.43 | −3.38 | −4.63 |
| | NOS | BREAST, NOS | | | | | | | | | | | |
| 668_s_at | BREAST, | MALIGNANT NEOPLASM OF FEMALE | INFILTRATING DUCT AND LOBULAR | 257.60 | 240.85 | 74 | 91.89 | 144.84 | 115.11 | 7 | 85.71 | −1.78 | −2.18 |
| | NOS | BREAST, NOS | CARCINOMA | | | | | | | | | | |
| 668_s_at | LARYNX, | MALIGNANT NEOPLASM OF LARYNX, | SQUAMOUS CELL CARCINOMA, NOS | 28.72 | 27.18 | 5 | 60.00 | 234.14 | 180.12 | 9 | 100.00 | 8.13 | 3.35 |
| | NOS | NOS | | | | | | | | | | | |
| 668_s_at | LUNG, NOS | MALIGNANT NEOPLASM | SQUAMOUS CELL CARCINOMA, NOS | 70.43 | 134.23 | 93 | 77.42 | 222.22 | 182.48 | 33 | 90.91 | 3.16 | 4.38 |
| | | OF LUNG, NOS | | | | | | | | | | | |
| 668_s_at | LUNG, NOS | MALIGNANT NEOPLASM | ADENOCARCINOMA, NOS | 70.43 | 134.23 | 93 | 77.42 | 168.03 | 220.33 | 36 | 77.78 | 2.39 | 2.49 |
| | | OF LUNG, NOS | | | | | | | | | | | |
| 668_s_at | STOMACH, | MALIGNANT NEOPLASM | ADENOCARCINOMA, NOS | 57.94 | 224.68 | 45 | 35.56 | 359.90 | 597.08 | 33 | 81.82 | 6.21 | 2.77 |
| | NOS | OF STOMACH, | | | | | | | | | | | |
| | | NOS | | | | | | | | | | | |
| 668_s_at | COLON, | SECONDARY MALIGNANT | ADENOCARCINOMA, NOS | 14.79 | 93.49 | 142 | 5.63 | 365.99 | 124.69 | 3 | 100.00 | 25.00 | 4.85 |
| | NOS | NEOPLASM OF | | | | | | | | | | | |
| | | COLON, NOS | | | | | | | | | | | |
| 668_s_at | COLON, | BENIGN NEOPLASM OF COLON, NOS | ADENOMA, NOS | 14.79 | 93.49 | 142 | 5.63 | 227.61 | 218.87 | 10 | 80.00 | 15.39 | 3.06 |
| | NOS | | | | | | | | | | | | |
| 668_s_at | COLON, | MALIGNANT NEOPLASM | MUCINOUS ADENOCARCINOMA | 14.79 | 93.49 | 142 | 5.63 | 328.81 | 239.51 | 8 | 75.00 | 22.22 | 3.69 |
| | NOS | OF COLON, NOS | | | | | | | | | | | |
| 668_s_at | COLON, | MALIGNANT NEOPLASM | ADENOCARCINOMA, NOS | 14.79 | 93.49 | 142 | 5.63 | 206.47 | 267.90 | 77 | 81.82 | 13.89 | 6.08 |
| | NOS | OF COLON, NOS | | | | | | | | | | | |
| 668_s_at | RECTUM, | MALIGNANT NEOPLASM OF RECTUM | ADENOCARCINOMA, NOS | 14.53 | 72.16 | 46 | 4.35 | 409.54 | 523.94 | 33 | 90.91 | 28.57 | 4.30 |
| | NOS | | | | | | | | | | | | |
| 668_s_at | LIVER, | CIRRHOSIS OF LIVER, NOS | FIBROSIS, NOS | 16.46 | 50.87 | 29 | 20.69 | 150.90 | 164.64 | 29 | 93.10 | 9.17 | 4.20 |
| | NOS | | | | | | | | | | | | |
| 668_s_at | LIVER, | SECONDARY MALIGNANT | ADENOCARCINOMA, NOS | 16.46 | 50.87 | 29 | 20.69 | 552.65 | 834.24 | 12 | 83.33 | 33.33 | 2.23 |
| | NOS | NEOPLASM OF | | | | | | | | | | | |
| | | LIVER, NOS | | | | | | | | | | | |
| 668_s_at | LIVER, | LIVER DISEASE, NOS | FOCAL NODULAR HYPERPLASIA | 16.46 | 50.87 | 29 | 20.69 | 150.04 | 105.77 | 4 | 75.00 | 9.09 | 2.49 |
| | NOS | | | | | | | | | | | | |
| 668_s_at | PAN- | MALIGNANT NEOPLASM | ADENOCARCINOMA, NOS | 540.07 | 528.88 | 20 | 100.00 | 1358.54 | 751.83 | 30 | 100.00 | 2.51 | 4.52 |
| | CREAS, | OF PANCREAS, | | | | | | | | | | | |
| | NOS | NOS | | | | | | | | | | | |
| 668_s_at | KIDNEY, | FOCAL GLOMERULOSCLEROSIS | GLOMERULOSCLEROSIS, NOS | 320.04 | 362.71 | 70 | 95.71 | 1401.05 | 214.60 | 3 | 100.00 | 4.39 | 8.24 |
| | NOS | | | | | | | | | | | | |
| 668_s_at | KIDNEY, | MALIGNANT NEOPLASM | TRANSITIONAL CELL CARCINOMA, | 320.04 | 362.71 | 70 | 95.71 | 26.43 | 27.85 | 4 | 50.00 | −12.11 | −6.45 |
| | NOS | OF KIDNEY, NOS | NOS | | | | | | | | | | |
| 668_s_at | KIDNEY, | DIABETIC NEPHROPATHY | GLOMERULOSCLEROSIS, NOS | 320.04 | 362.71 | 70 | 95.71 | 1276.45 | 541.70 | 3 | 100.00 | 3.98 | 3.03 |
| | NOS | | | | | | | | | | | | |
| 668_s_at | KIDNEY, | BENIGN NEOPLASM OF KIDNEY, NOS | ONCOCYTOMA | 320.04 | 362.71 | 70 | 95.71 | 86.57 | 110.11 | 6 | 66.67 | −3.70 | −3.74 |
| | NOS | | | | | | | | | | | | |
| 668_s_at | BLADDER, | MALIGNANT NEOPLASM | TRANSITIONAL CELL CARCINOMA, | 13.59 | 15.34 | 6 | 33.33 | 467.71 | 307.33 | 4 | 100.00 | 34.48 | 2.95 |
| | NOS | OF BLADDER, | NOS | | | | | | | | | | |
| | | NOS | | | | | | | | | | | |
| 668_s_at | VULVA, | MALIGNANT NEOPLASM | SQUAMOUS CELL CARCINOMA, NOS | 31.96 | 19.63 | 3 | 100.00 | 285.52 | 366.85 | 10 | 80.00 | 8.93 | 2.18 |
| | NOS | OF VULVA, NOS | | | | | | | | | | | |
| 668_s_at | ENDO- | MALIGNANT NEOPLASM OF | PAPILLARY SEROUS | 444.86 | 558.15 | 26 | 96.15 | 967.86 | 454.04 | 4 | 100.00 | 2.17 | 2.08 |
| | METRIUM, | ENDOMETRIUM | ADENOCARCINOMA | | | | | | | | | | |
| | NOS | | | | | | | | | | | | |
| 668_s_at | ENDO- | MALIGNANT NEOPLASM OF | ADENOCARCINOMA, NOS | 444.86 | 558.15 | 26 | 96.15 | 875.53 | 934.03 | 62 | 98.39 | 1.97 | 2.67 |
| | METRIUM, | ENDOMETRIUM | | | | | | | | | | | |
| | NOS | | | | | | | | | | | | |
| 668_s_at | MYO- | ADENOMYOSIS | ENDOMETRIOSIS, NOS | 114.68 | 118.46 | 120 | 84.17 | 388.83 | 190.06 | 7 | 100.00 | 3.39 | 3.77 |
| | METRIUM, | | | | | | | | | | | | |
| | NOS | | | | | | | | | | | | |
| 668_s_at | OVARY, | MALIGNANT NEOPLASM OF OVARY | PAPILLARY SEROUS | 4.61 | 27.04 | 68 | 11.76 | 525.51 | 580.08 | 32 | 100.00 | 111.11 | 5.08 |
| | NOS | | ADENOCARCINOMA | | | | | | | | | | |
| 668_s_at | OVARY, | MALIGNANT NEOPLASM OF OVARY | SEROUS CYSTADENOCARCINOMA, | 4.61 | 27.04 | 68 | 11.76 | 635.15 | 572.42 | 6 | 83.33 | 142.86 | 2.70 |
| | NOS | | NOS | | | | | | | | | | |
| 668_s_at | OVARY, | MALIGNANT NEOPLASM OF OVARY | CLEAR CELL ADENOCARCINOMA, | 4.61 | 27.04 | 68 | 11.76 | 396.62 | 486.20 | 8 | 75.00 | 83.33 | 2.28 |
| | NOS | | NOS | | | | | | | | | | |
| 668_s_at | OVARY, | MALIGNANT NEOPLASM OF OVARY | ADENOCARCINOMA, NOS | 4.61 | 27.04 | 68 | 11.76 | 800.39 | 1067.94 | 12 | 83.33 | 166.67 | 2.58 |
| | NOS | | | | | | | | | | | | |
| 668_s_at | OVARY, | SECONDARY MALIGNANT | ADENOCARCINOMA, NOS | 4.61 | 27.04 | 68 | 11.76 | 725.08 | 602.71 | 6 | 100.00 | 166.67 | 2.93 |
| | NOS | NEOPLASM OF | | | | | | | | | | | |
| | | OVARY | | | | | | | | | | | |
| 668_s_at | OVARY, | NEOPLASM OF UNCERTAIN | SEROUS CYSTADENOMA, | 4.61 | 27.04 | 68 | 11.76 | 768.87 | 267.40 | 3 | 100.00 | 166.67 | 4.95 |
| | NOS | BEHAVIOR | BORDERLINE MALIGNANCY | | | | | | | | | | |
| | | OF OVARY | | | | | | | | | | | |
| 668_s_at | PROSTATE, | MALIGNANT NEOPLASM | ADENOCARCINOMA, NOS | 389.66 | 332.84 | 33 | 96.97 | 232.08 | 340.03 | 49 | 93.88 | −1.68 | −2.08 |
| | NOS | OF PROSTATE | | | | | | | | | | | |
| 668_s_at | OMEN- | SECONDARY MALIGNANT | PAPILLARY SEROUS | 22.82 | 38.44 | 15 | 40.00 | 414.09 | 445.89 | 24 | 100.00 | 18.18 | 4.27 |
| | TUM, NOS | NEOPLASM OF | ADENOCARCINOMA | | | | | | | | | | |
| | | THE OMENTUM | | | | | | | | | | | |

¹Refers to control or normal sample set.
²Refers to experimental or diseased sample set.

TABLE 4
Differential Gene Expression with Respect to Staged Disease/Morphology

| | | | | | | C¹ | | | CPC | | | | | Fold | |
|---|---|---|---|---|---|---|---|---|---|---|---|---|---|---|---|
| Affy ID | Donor Disease | Tissue | Disease | Morphology | Stage | Mean | C SD | C # | % | E²Mean | E SD | E # | EPC % | Change | T-Stat |
| 668_s_at | MALIGNANT | LUNG, NOS | MALIGNANT NEOPLASM | SQUAMOUS CELL | T2N0MX | 55.16 | 69.43 | 53 | 79.25 | 227.35 | 207.13 | 13 | 84.62 | 4.12 | 2.96 |
| | NEOPLASM | | OF LUNG, NOS | CARCINOMA, NOS | | | | | | | | | | | |
| | OF LUNG, | | | | | | | | | | | | | | |
| | NOS | | | | | | | | | | | | | | |
| 668_s_at | MALIGNANT | LUNG, NOS | MALIGNANT NEOPLASM | SQUAMOUS CELL | T2N1MX | 55.16 | 69.43 | 53 | 79.25 | 332.21 | 227.80 | 4 | 100.00 | 6.02 | 2.42 |
| | NEOPLASM | | OF LUNG, NOS | CARCINOMA, NOS | | | | | | | | | | | |
| | OF LUNG, | | | | | | | | | | | | | | |
| | NOS | | | | | | | | | | | | | | |
| 668_s_at | MALIGNANT | LUNG, NOS | MALIGNANT NEOPLASM | SQUAMOUS CELL | T1N0MX | 55.16 | 69.43 | 53 | 79.25 | 187.89 | 113.06 | 6 | 100.00 | 3.40 | 2.82 |
| | NEOPLASM | | OF LUNG, NOS | CARCINOMA, NOS | | | | | | | | | | | |
| | OF LUNG, | | | | | | | | | | | | | | |
| | NOS | | | | | | | | | | | | | | |
| 668_s_at | MALIGNANT | LUNG, NOS | MALIGNANT NEOPLASM | ADENOCARCINOMA, | T1N0MX | 55.16 | 69.43 | 53 | 79.25 | 236.21 | 320.57 | 11 | 81.82 | 4.27 | 1.86 |
| | NEOPLASM | | OF LUNG, NOS | NOS | | | | | | | | | | | |
| | OF LUNG, | | | | | | | | | | | | | | |
| | NOS | | | | | | | | | | | | | | |
| 668_s_at | MALIGNANT | COLON, NOS | MALIGNANT NEOPLASM | ADENOCARCINOMA, | T3N2M0 | 21.74 | 109.71 | 53 | 7.55 | 322.66 | 139.89 | 3 | 100.00 | 14.93 | 3.66 |
| | NEOPLASM | | OF COLON, NOS | NOS | | | | | | | | | | | |
| | OF COLON, | | | | | | | | | | | | | | |
| | NOS | | | | | | | | | | | | | | |
| 668_s_at | MALIGNANT | COLON, NOS | MALIGNANT NEOPLASM | ADENOCARCINOMA, | T3N2MX | 21.74 | 109.71 | 53 | 7.55 | 342.27 | 420.24 | 7 | 85.71 | 15.63 | 2.01 |
| | NEOPLASM | | OF COLON, NOS | NOS | | | | | | | | | | | |
| | OF COLON, | | | | | | | | | | | | | | |
| | NOS | | | | | | | | | | | | | | |
| 668_s_at | MALIGNANT | COLON, NOS | MALIGNANT NEOPLASM | ADENOCARCINOMA, | T3N0M0 | 21.74 | 109.71 | 53 | 7.55 | 266.08 | 127.10 | 3 | 100.00 | 12.20 | 3.26 |
| | NEOPLASM | | OF COLON, NOS | NOS | | | | | | | | | | | |
| | OF COLON, | | | | | | | | | | | | | | |
| | NOS | | | | | | | | | | | | | | |
| 668_s_at | MALIGNANT | COLON, NOS | MALIGNANT NEOPLASM | ADENOCARCINOMA, | T3N0MX | 21.74 | 109.71 | 53 | 7.55 | 208.67 | 245.70 | 13 | 84.62 | 9.62 | 2.68 |
| | NEOPLASM | | OF COLON, NOS | NOS | | | | | | | | | | | |
| | OF COLON, | | | | | | | | | | | | | | |
| | NOS | | | | | | | | | | | | | | |
| 668_s_at | MALIGNANT | RECTUM, | MALIGNANT NEOPLASM | ADENOCARCINOMA, | T3N0M0 | 18.94 | 78.59 | 24 | 4.17 | 337.00 | 182.06 | 3 | 100.00 | 17.86 | 2.99 |
| | NEOPLASM | NOS | OF RECTUM | NOS | | | | | | | | | | | |
| | OF RECTUM | | | | | | | | | | | | | | |
| 668_s_at | MALIGNANT | KIDNEY, | MALIGNANT NEOPLASM | CLEAR CELL | T2NXMX | 304.28 | 402.59 | 37 | 91.89 | 74.29 | 81.28 | 4 | 75.00 | −4.10 | −2.96 |
| | NEOPLASM | NOS | OF KIDNEY, NOS | ADENOCARCINOMA, | | | | | | | | | | | |
| | OF KIDNEY, | | | NOS | | | | | | | | | | | |
| | NOS | | | | | | | | | | | | | | |
| 668_s_at | MALIGNANT | PROSTATE, | MALIGNANT NEOPLASM | ADENOCARCINOMA, | T3AN0MX | 373.35 | 307.25 | 27 | 96.30 | 63.58 | 77.60 | 8 | 75.00 | −5.87 | −4.75 |
| | NEOPLASM | NOS | OF PROSTATE | NOS | | | | | | | | | | | |
| | OF | | | | | | | | | | | | | | |
| | PROSTATE | | | | | | | | | | | | | | |
| 668_s_at | MALIGNANT | PROSTATE, | MALIGNANT NEOPLASM | ADENOCARCINOMA, | T2BNXMX | 373.35 | 307.25 | 27 | 96.30 | 212.61 | 185.48 | 7 | 100.00 | −1.76 | −1.75 |
| | NEOPLASM | NOS | OF PROSTATE | NOS | | | | | | | | | | | |
| | OF | | | | | | | | | | | | | | |
| | PROSTATE | | | | | | | | | | | | | | |
| 668_s_at | MALIGNANT | PROSTATE, | MALIGNANT NEOPLASM | ADENOCARCINOMA, | T2AN0MX | 373.35 | 307.25 | 27 | 96.30 | 156.88 | 160.27 | 6 | 100.00 | −2.38 | −2.46 |
| | NEOPLASM | NOS | OF PROSTATE | NOS | | | | | | | | | | | |
| | OF | | | | | | | | | | | | | | |
| | PROSTATE | | | | | | | | | | | | | | |
| 668_s_at | MALIGNANT | OMENTUM, | SECONDARY | PAPILLARY SEROUS | T3BNXMX | 14.04 | 14.04 | 5 | 40.00 | 361.90 | 284.60 | 4 | 100.00 | 25.64 | 2.44 |
| | NEOPLASM | NOS | MALIGNANT | ADENOCARCINOMA | | | | | | | | | | | |
| | OF OVARY | | NEOPLASM OF THE | | | | | | | | | | | | |
| | | | OMENTUM | | | | | | | | | | | | |
| 668_s_at | MALIGNANT | OMENTUM, | SECONDARY | PAPILLARY SEROUS | T3CNXMX | 14.04 | 14.04 | 5 | 40.00 | 274.69 | 222.99 | 6 | 100.00 | 19.61 | 2.86 |
| | NEOPLASM | NOS | MALIGNANT | ADENOCARCINOMA | | | | | | | | | | | |
| | OF OVARY | | NEOPLASM OF THE | | | | | | | | | | | | |
| | | | OMENTUM | | | | | | | | | | | | |
| 668_s_at | MALIGNANT | BREAST, | MALIGNANT NEOPLASM | INTRADUCTAL | TISNXMX | 283.68 | 233.67 | 29 | 89.66 | 124.69 | 84.31 | 3 | 100.00 | −2.28 | −2.44 |
| | NEOPLASM | NOS | OF FEMALE BREAST, NOS | CARCINOMA, NOS | | | | | | | | | | | |
| | OF FEMALE | | | | | | | | | | | | | | |
| | BREAST, NOS | | | | | | | | | | | | | | |
| 668_s_at | MALIGNANT | BREAST, | MALIGNANT NEOPLASM | INFILTRATING DUCT | T3N1M0 | 283.68 | 233.67 | 29 | 89.66 | 117.48 | 73.03 | 3 | 100.00 | −2.42 | −2.75 |
| | NEOPLASM | NOS | OF FEMALE BREAST, NOS | CARCINOMA | | | | | | | | | | | |
| | OF FEMALE | | | | | | | | | | | | | | |
| | BREAST, NOS | | | | | | | | | | | | | | |
| 668_s_at | MALIGNANT | BREAST, | MALIGNANT NEOPLASM | INFILTRATING DUCT | T2N0M0 | 283.68 | 233.67 | 29 | 89.66 | 149.03 | 139.39 | 9 | 100.00 | −1.90 | −2.12 |
| | NEOPLASM | NOS | OF FEMALE BREAST, NOS | CARCINOMA | | | | | | | | | | | |
| | OF FEMALE | | | | | | | | | | | | | | |
| | BREAST, NOS | | | | | | | | | | | | | | |
| 668_s_at | MALIGNANT | BREAST, | MALIGNANT NEOPLASM | INFILTRATING DUCT | T2N0MX | 283.68 | 233.67 | 29 | 89.66 | 28.70 | 21.84 | 8 | 62.50 | −9.89 | −5.79 |
| | NEOPLASM | NOS | OF FEMALE BREAST, NOS | CARCINOMA | | | | | | | | | | | |
| | OF FEMALE | | | | | | | | | | | | | | |
| | BREAST, NOS | | | | | | | | | | | | | | |
| 668_s_at | MALIGNANT | BREAST, | MALIGNANT NEOPLASM | INFILTRATING DUCT | T3N2M0 | 283.68 | 233.67 | 29 | 89.66 | 22.97 | 25.11 | 4 | 50.00 | −12.35 | −5.77 |
| | NEOPLASM | NOS | OF FEMALE BREAST, NOS | CARCINOMA | | | | | | | | | | | |
| | OF FEMALE | | | | | | | | | | | | | | |
| | BREAST, NOS | | | | | | | | | | | | | | |

¹Denotes control set composed of normal samples of the listed tissue type from patients with the indicated donor disease.
²Denotes experimental set composed of samples of the listed tissue, disease, morphology and disease stage from patients with the indicated donor disease.

TABLE 5
Differential Gene Expression in Normal Tissues with Respect to Donor's Secondary Disease

| | | | | | | | | | | | Fold | |
|---|---|---|---|---|---|---|---|---|---|---|---|---|
| Affy ID | Tissue | SecDisease | C¹Mean | C SD | C # | CPC % | E²Mean | E SD | E # | EPC % | Change | T-Stat |
| 668_s_at | PANCREAS, | MALIGNANT NEOPLASM | 400.47 | 406.24 | 17 | 100.00 | 1331.16 | 482.32 | 3 | 100.00 | 3.32 | 3.15 |
| | NOS | OF PANCREAS, | | | | | | | | | | |
| | | NOS | | | | | | | | | | |
| 668_s_at | KIDNEY, NOS | HYPOTHYROIDISM, NOS | 331.13 | 366.89 | 67 | 95.52 | 72.52 | 23.17 | 3 | 100.00 | −4.57 | −5.53 |
| 668_s_at | UTERUS, NOS | GASTROESOPHAGEAL | 262.87 | 339.03 | 45 | 91.11 | 136.55 | 84.52 | 4 | 75.00 | −1.93 | −1.92 |
| | | REFLUX | | | | | | | | | | |
| | | DISEASE | | | | | | | | | | |
| 668_s_at | UTERUS, NOS | ADENOMYOSIS | 264.85 | 338.09 | 45 | 91.11 | 114.37 | 91.33 | 4 | 75.00 | −2.32 | −2.21 |
| 668_s_at | UTERUS, NOS | MALIGNANT NEOPLASM | 272.11 | 338.88 | 44 | 90.91 | 80.50 | 92.85 | 5 | 80.00 | −3.38 | −2.91 |
| | | OF UTERINE | | | | | | | | | | |
| | | CERVIX | | | | | | | | | | |
| 668_s_at | CERVIX, NOS | MALIGNANT NEOPLASM | 80.44 | 140.32 | 64 | 73.44 | 156.94 | 239.12 | 36 | 83.33 | 1.95 | 1.76 |
| | | OF ENDOMETRIUM | | | | | | | | | | |
| 668_s_at | FALLOPIAN | HYPERTENSION, NOS | 353.26 | 538.27 | 26 | 92.31 | 137.55 | 115.83 | 8 | 75.00 | −2.57 | −1.91 |
| | TUBE, NOS | | | | | | | | | | | |
| 668_s_at | FALLOPIAN | CHRONIC CERVICITIS | 320.71 | 499.81 | 31 | 87.10 | 114.41 | 75.86 | 3 | 100.00 | −2.80 | −2.07 |
| | TUBE, NOS | | | | | | | | | | | |

¹Denotes control set composed of normal samples of the listed tissue type from donors not suffering from the indicated secondary disease.
²Denotes experimental set composed of normal samples of the listed tissue type from donors suffering from the indicated secondary disease.

TABLE 6
Differential Gene Expression in Normal Tissues with Respect to Medication

| Affy ID | Tissue | Medication | C¹Mean | C SD | C # | CPC % | E²Mean | E SD | E # | EPC % | Fold Change | T-Stat |
|---|---|---|---|---|---|---|---|---|---|---|---|---|
| 668_s_at | BREAST, NOS | Oral contraceptive | 266.24 | 244.36 | 70 | 92.86 | 106.53 | 77.43 | 4 | 75.00 | −2.50 | −3.29 |
| | | preparation (substance) | | | | | | | | | | |
| 668_s_at | KIDNEY, NOS | Metoprolol (substance) | 326.05 | 369.32 | 67 | 95.52 | 176.41 | 81.35 | 4 | 100.00 | −1.85 | −2.46 |
| 668_s_at | KIDNEY, NOS | Glyburide (substance) | 324.54 | 367.08 | 68 | 95.59 | 160.66 | 26.45 | 3 | 100.00 | −2.02 | −3.48 |
| 668_s_at | KIDNEY, NOS | Cardizem SR | 326.44 | 369.15 | 67 | 95.52 | 169.87 | 81.56 | 4 | 100.00 | −1.92 | −2.58 |
| | | Capsules (substance) | | | | | | | | | | |
| 668_s_at | KIDNEY, NOS | Zantac 150 | 327.97 | 365.04 | 68 | 97.06 | 83.09 | 56.29 | 3 | 66.67 | −3.95 | −4.46 |
| | | Tablets (substance) | | | | | | | | | | |
| 668_s_at | KIDNEY, NOS | Flomax (substance) | 325.75 | 366.33 | 68 | 95.59 | 133.24 | 66.35 | 3 | 100.00 | −2.45 | −3.28 |

¹Denotes control set composed of normal samples of the listed tissue type from patients not receiving the indicated medication.
²Denotes experimental set composed of normal samples of the listed tissue type from patients receiving the indicated medication.

TABLE 7
Differential Gene Expression in Normal Tissues with Respect to Age

| | | | | | | | | | | | Fold | |
|---|---|---|---|---|---|---|---|---|---|---|---|---|
| Affy ID | Tissue | AgeGroup | C¹Mean | C SD | C # | CPC % | E²Mean | E SD | E # | EPC % | Change | T-Stat |
| 668_s_at | KIDNEY, NOS | 40-50 yrs | 345.66 | 386.79 | 59 | 94.92 | 182.65 | 124.09 | 11 | 100.00 | −1.89 | −2.60 |
| 668_s_at | KIDNEY, NOS | 50-60 yrs | 372.27 | 399.69 | 52 | 96.15 | 169.17 | 149.19 | 18 | 94.44 | −2.20 | −3.09 |
| 668_s_at | KIDNEY, NOS | 70-80 yrs | 281.82 | 375.23 | 54 | 94.44 | 449.05 | 290.97 | 16 | 100.00 | 1.59 | 1.88 |

¹Denotes control set composed of normal tissues of the listed tissue type from patients other than the age group indicated.
²Denotes experimental set composed of normal tissues of the listed tissue type specifically from the indicated age group.

TABLE 8
Differential Gene Expression in Normal Tissues with Respect to Race

| Affy ID | Tissue | Race | C¹Mean | C SD | C # | CPC % | E²Mean | E SD | E # | EPC % | Fold Change | T-Stat |
|---|---|---|---|---|---|---|---|---|---|---|---|---|
| 668_s_at | BREAST, NOS | BLACK OR AFRICAN | 273.31 | 241.76 | 69 | 94.20 | 42.41 | 52.08 | 4 | 50.00 | −6.44 | −5.91 |
| | | AMERICAN | | | | | | | | | | |
| 668_s_at | FALLOPIAN | BLACK OR AFRICAN | 466.38 | 674.88 | 15 | 93.33 | 95.03 | 86.69 | 7 | 71.43 | −4.91 | −2.09 |
| | TUBE, NOS | AMERICAN | | | | | | | | | | |

¹Denotes control set composed of normal samples of the listed tissue type from patients other than the racial group indicated.
²Denotes experimental set composed of normal samples of the listed tissue type specifically from the indicated racial group.

TABLE 9
Differential Gene Expression in Normal Tissues with Respect to Gender
NO SIGNIFICANT DIFFERENTIAL GENE EXPRESSION

Although the present invention has been described in detail with reference to the example above, it is understood that various modifications can be made without departing from the spirit of the invention. Accordingly, the invention is limited only by the following claims. All cited patents, patent applications, and publications referred to in this application are herein incorporated by reference in their entirety.

What is claimed:

1. A method of providing one or more gene annotation reports to a customer comprising:
a. receiving at least one gene identifier for a gene from a customer;
b. interrogating one or more databases with the gene identifier;
c. producing a gene annotation report for the gene; and
d. forwarding the gene annotation report to the customer.

2. The method of claim 1, wherein one or more of said databases is privately owned.

3. The method of claims 1 or 2 wherein the customer is not a subscriber to at least one of the privately owned databases.

4. The method of claim 1, wherein the gene annotation report is provided through a gene annotation database.

5. The method of claim 4, wherein the gene annotation database is a gene expression database.

6. The method of claim 4 wherein the gene annotation database comprises multiple databases.

7. The method of claim 4, wherein the gene annotation database uses an algorithm employing a hierarchical method for organizing biological samples for analysis using a b-tree and query grammar to manage and explore gene expression and related data.

8. The method of claim 6, wherein at least one of said multiple databases employs a sequence alignment algorithm.

9. The method of claim 1, wherein the gene identifier is selected from the group consisting of a nucleotide sequence, an amino acid sequence, a sequence database identifier, a gene name, a protein name, a disease name and a reference citation.

10. The method of claim 9, wherein the sequence database identifier is selected from the group consisting of a GenBank accession number and an Affymetrix™ fragment identifier.

11. The method of claim 1, wherein the customer is a one-time user.

12. The method of claim 1, wherein at least one gene identifier is received from the customer electronically.

13. The method of claim 12, wherein at least one gene identifier is received from the customer using an electronic delivery means selected from the group consisting of electronic mail, an internet download, a modem-to-modem download, and a computer-readable storage medium.

14. The method of claim 1, wherein the gene annotation report comprises information concerning the identity of the cell or tissue wherein the gene is expressed.

15. The method of claim 14, wherein the gene annotation report further comprises information concerning gene expression levels in more than one cell or tissues.

16. The method of claim 14, wherein the information concerning the identity of the cell or tissue is the disease state of the cell or tissue, a physiological characteristic of the cell or tissue, and/or information concerning the patient from whom the cell or tissue was derived.

17. The method of claim 14, wherein the gene annotation report further comprises genomic information selected from the group consisting of clone expression, single nucleotide polymorphisms, splice variants, functional and/or structural domains, promoter sequences, transcription elements, map location, known alleles and/or mutants, molecular weight, cleavage sites, biological pathways or diseases in which the gene is involved, ligands, antibodies, relevant pharmaceuticals, gene family relationships and the locations of clones, specialists and relevant clinical trials.

18. The method of claim 1, wherein interrogating a gene expression database with the gene identifier comprises:
(i) comparing the gene identifier to information in the gene expression database; and
(ii) incorporating results of the comparison into the gene annotation report.

19. The method of claim 18, wherein the gene identifier is a sequence and the comparing comprises the step of comparing the sequence to sequences in the sequence database.

20. The method of claim 18, wherein the comparing comprises aligning or calculating sequence homology.

21. The method of claim 18, wherein the gene identifier is an accession number and the comparing step comprises locating the accession number in the sequence database.

22. The method of claim 1, wherein the forwarding of step (d) is done electronically.

23. The method of claim 22, wherein the forwarding electronically uses a delivery means selected from the group consisting of electronic mail, an internet download, a modem-to-modem download, and a computer-readable storage medium.

24. The method of claim 1, wherein two or more gene identifiers are received from a customer.

25. The method of claim 24, wherein said two or more gene identifiers relate to a family or subset of biologically and/or functionally related genes.

26. The method of claim 25, wherein said family or subset of related genes is selected from the group consisting of families or subsets of genes involved in one or more biological or signal transduction pathways, genes encoding homologous proteins, genes encoding proteins that share conserved motifs, genes that encode the top pharmaceutical drug targets and genes involved in a specified disease.

27. The method of claim 26, wherein said genes encoding proteins that share conserved motifs are selected from the group consisting of genes encoding G-protein coupled receptors, kinases, antibodies and DNA binding proteins.

28. A gene annotation report provided by the method of claim 1.

29. The gene annotation report of claim 28, wherein the gene annotation report comprises information concerning the identity of the cell or tissue wherein the gene is expressed.

30. The gene annotation report of claim 29, wherein the gene annotation report further comprises information concerning gene expression levels in more than one cell or tissues.

31. The gene annotation report of claim 29, wherein the information concerning the identity of the cell or tissue is the disease state of the cell or tissue, a physiological characteristic of the cell or tissue, and/or information concerning the patient from whom the cell or tissue was derived.

32. The gene annotation report of claim 28, wherein the gene annotation report further comprises genomic information selected from the group consisting of clone expression, single nucleotide polymorphisms, splice variants, functional and/or structural domains, promoter sequences, transcription elements, map location, known alleles and/or mutants, molecular weight, cleavage sites, biological pathways or diseases in which the gene is involved, ligands, antibodies, relevant pharmaceuticals, gene family relationships and the locations of clones, specialists and relevant clinical trials.
